SEQUENCE LISTING (6 sequences)

SEQ ID NO: 1 - DNA, 1959 bp, Escherichia coli
  source 1..1959: mol_type = other DNA
atgagccaaattcacaaacacaccattcctgccaacatcgcagaccgttgcctgataaaccctcagcagtacgaggcgatgtatcaacaatctattaacgtacctgataccttctggggcgaacagggaaaaattcttgactggatcaaaccttaccagaaggtgaaaaacacctcctttgcccccggtaatgtgtccattaaatggtacgaggacggcacgctgaatctggcggcaaactgccttgaccgccatctgcaagaaaacggcgatcgtaccgccatcatctgggaaggcgacgacgccagccagagcaaacatatcagctataaagagctgcaccgcgacgtctgccgcttcgccaataccctgctcgagctgggcattaaaaaaggtgatgtggtggcgatttatatgccgatggtgccggaagccgcggttgcgatgctggcctgcgcccgcattggcgcggtgcattcggtgattttcggcggcttctcgccggaagccgttgccgggcgcattattgattccaactcacgactggtgatcacttccgacgaaggtgtgcgtgccgggcgcagtattccgctgaagaaaaacgttgatgacgcgctgaaaaacccgaacgtcaccagcgtagagcatgtggtggtactgaagcgtactggcgggaaaattgactggcaggaagggcgcgacctgtggtggcacgacctggttgagcaagcgagcgatcagcaccaggcggaagagatgaacgccgaagatccgctgtttattctctacacctccggttctaccggtaagccaaaaggtgtgctgcatactaccggcggttatctggtgtacgcggcgctgacctttaaatatgtctttgattatcatccgggtgatatctactggtgcaccgccgatgtgggctgggtgaccggacacagttacttgctgtacggcccgctggcctgcggtgcgaccacgctgatgtttgaaggcgtacccaactggccgacgcctgcccgtatggcgcaggtggtggacaagcatcaggtcaatattctctataccgcacccacggcgatccgcgcgctgatggcggaaggcgataaagcgatcgaaggcaccgaccgttcgtcgctgcgcattctcggttccgtgggcgagccaattaacccggaagcgtgggagtggtactggaaaaaaatcggcaacgagaaatgtccggtggtcgatacctggtggcagaccgaaaccggcggtttcatgatcaccccgctgcctggcgctaccgagctgaaagccggttcggcaacacgtccgttcttcggcgtgcaaccggcgctggtcgataacgaaggtaacccgctggagggggccaccgaaggtagcctggtaatcaccgactcctggccgggtcaggcgcgtacgctgtttggcgatcacgaacgttttgaacagacctacttctccaccttcaaaaatatgtatttcagcggcgacggcgcgcgtcgcgatgaagatggctattactggataaccgggcgtgtggacgacgtgctgaacgtctccggtcaccgtctggggacggcagagattgagtcggcgctggtggcgcatccgaagattgccgaagccgccgtagtaggtattccgcacaatattaaaggtcaggcgatctacgcctacgtcacgcttaatcacggggaggaaccgtcaccagaactgtacgcagaagtccgcaactgggtgcgtaaagagattggcccgctggcgacgccagacgtgctgcactggaccgactccctgcctaaaacccgctccggcaaaattatgcgccgtattctgcgcaaaattgcggcgggcgataccagcaacctgggcgatacctcgacgcttgccgatcctggcgtagtcgagaagctgcttgaagagaagcaggctatcgcgatgccatcgtaa

SEQ ID NO: 2 - DNA, 510 bp, Escherichia coli
  source 1..510: mol_type = other DNA
atgcgtcatgtagagcatacagtcaccgttgcggccccagcagacttggtttgggaggtacttgccgatgtcttaggctatgctgacatcttcccaccgacggaaaaagttgaaattcttgaggaggggcaaggataccaggtagtgcgccttcacgtcgatgttgcgggtgagattaatacatggaccagtcgtcgcgatttagaccctgcgcgccgcgtaattgcttaccgccaacttgagacggctccgatcgtgggccacatgagcggggaatggcgtgctttcacactggatgccgaacgtacccaattagtcctgactcacgatttcgtaacccgtgcagccggggatgacggtttagtcgccggaaaattgaccccagatgaggcgcgcgaaatgttagaagcggtggtagaacgtaactctgtcgccgacttaaacgcggtccttggagaagctgagcgtcgcgtccgcgcagccggtggagttggtaccgtaactgcgtaa

SEQ ID NO: 3 - DNA, 771 bp, Escherichia coli
  source 1..771: mol_type = other DNA
atgtcagggcgcaaaacctttttagacttaagttttgctacccgcgacacaccgtcggaggcgactccggtggtggtagatttgctggaccacgtaactggagccaccgtattaggattatcacctgaggatttccccgatggtatggctatttccaatgagaccgttacgttgacgacccacactggcacgcacatggatgcgccactgcactatggtcccttaagtgggggagttccggcaaagtcgattgaccaagtgcccttggaatggtgctatggacctggagttcgtttggatgttcgccacgtgccggcaggagatggtattactgtcgatcatttgaacgccgcgttggatgcagcagagcacgatttggcccccggtgacattgtgatgctgtggaccggcgcggacgctctgtggggaacccgcgaatacttgagcacgtttccggggttaactgggaaggggacacaatttttggtcgaggcgggtgttaaagtcattggcattgatgcatggggactggatcgcccgatggcagctatgatcgaagaataccgtcgtacgggcgataaaggagcattatggccggctcacgtctatggacgcacacgcgaatacctgcaattagagaagcttaataatttgggcgctttaccaggagctacagggtatgacatttcatgctttccggttgcggttgcaggcactggagctgggtggactcgtgtggtcgccgttttcgagcaagaggaagaggattaa

SEQ ID NO: 4 - DNA, 6425 bp, Escherichia coli
  source 1..6425: mol_type = other DNA
atgataataaataacagaaatgaatctcaaccacgtagagttgtggtgacagggctaggtgttgtcgcaccgacaggtgttggcgttaatgaattttggaacaatattcataacggcaaatcgggggtaagtgaatatgagtggggaagaaaaaaatttggttttaaaagcggagcaataggaaaagttcacggtaacgatagcgatagcaaagagtttgtgctgaaaagtgagcgtaaatatcttgagtttgcgctagaagcctctgagatggcaatgcaagatgcaaatttaaaaccttcagacattgatggccggcgttttggcgttgcgatagcaacagcgattgccgatgctgcgggaatggaagagtgtttgctcaggatcaccaaagggggcaaagagaatattcatcctgatttaattaaatcagaggattatgacagctttgatttcagctctgccgccacctctgttgcgaaaaaatatggcgcatcgatgtccgtcagtaacatatcaactgggtgtgcggcaggacttgatgcattaggcattgcgatggagcatatccgttatggcagagcggatgtgatgctggctggcgccagtgaagcgccgctttgtccactttctatcggctcttttgaagctttaggggcgctatcatcaagagaattggaaaatcagcaagcagcgacttgtcctttttcccttgagcgggatggatttgtgattgctgaagggtgtggaatattaattttagagtcttatgaacatgctaagcagcgtggagcacatatctatgctgaattagcagggtatgcgtccgtgaataacgcttatcatatgaccgacttgcctgcggatggaatggcaatggcgcggtgcattgatatggcgttgaaggatgcccagatatcgccatcagcggtcaattatattagtgctcatggcagttctacggctcaaaatgatattaacgaatcaaatgcgattaaatttgttttgggagaaaatgcatttgatattccaattaactcattaaagtcaatgacaggtcatgctttagctgccgctaatgcgatcgagtctgtagcgttatgtctggaaatagaaaagcaatatattcatccaacaattaattatcaaacgccggaccctgattgcgatttagattatattcctaatcaaggttgcgcatatccaattaagaccgcattaaaattatcgagtggtttttctggtattcacagtgttattgttatgagggcagtagacaatgcgtaaaagagttgttgttaccggcgttggcgcagtacatcctgatggcaatgatgtcaccgctataaaaacaaaagtgattcagaaattattgggtcaggaatcgataaataataccaacaaaagttctgtaataaggacattgaatgatttcgatggggcaaaatatatcaataaccgcttaagacgtaaaattgatgaattttcagtttatggtatcgtcgccgttgaaatggcattaaaagcgagcagattggatgtagataagcttgatcctaatcgtgttggcatatatgttggaaactgttttggcggatggcagcatattgaggatgaagttaaagcgctccatgttgaaggcatatcggggatgggaccttatgttgctacggcatggttccctgctgcgcttcaagggcaattgtcactgctttatggttttagtgcgcaatctaagacattttccacctccgatgtagcagggatgcaagcaataggctatgcggctgaagcgatttctaatggtgttgccgaagtgatgttatgtggcgcgtcagaacatctttccagcccgttagttaaaagtttactggagaaagagtcaagccagaaacactctgaggtttttggcgaaagacagccaggggacttttccgaaggcgctgcatttctagtgctggaagagaggcaacatgctttagaacgcggcgcttcgatattgtgtgaattaacgggttttgttgattatttttcaccggataaaaatacaagaaataacaccttagaatatactgctgaactattcaaccataatgagaatgctgtatttattatggatggaatatatgatgatgaaaaagaaataacgagtaaggctttctccaataaagagataaaaacatcatttataaatctgaggccttacttgaataatcaattttcagtcagcggcgtaattgattcagtcctggcatcatcatttttatcagaaaataacggggatggagaacaacaatctaataaaataaatgaactttcaaatactaaccaaataataattcagcgctttagtaaccagggtcatgtatgtgcgttgagtttttcagcaatttaatctctaaaatatttaattacgcgaggaaaaatatatgaataataacccagaagtaaaaataaaaacgattttgtctctttttcttaacgttaatattgatgatttcaatatggatgcaaaccttgctgatgcctatgatatggattctacggaattggctgacttggcaaaagagattacgaaagagttcggtatttccgtgacgaaaagtcagttcagtcattgggaaacaggaagagccgttcttgatttcgtctcatcaagtttaaacgataaaaattaattgctaattaaagggaaacagaaatgaaactaatctctatgttgttacattcagagcatgataacttacatcatgattgtattgtcactaaggattatcattatacaagaaaagaggtgatatcttctgtttcccatttaattgatgatttattgagtcgaggagtgcaaaaaggtaataaagtcattgttatatttgaacatgatgaattaggtgttttctttttggctgccgccagtgctatggggttgcatttattaatgccctataatttatcatcagcgacaatcgatgaatggattaattttaccaatgaagtgcaatacgattttgttgtttatctcaaaaaagataaacattttgttggaaaattaaaagaaaacaacattaatgttattgatatttcagatcataagatcagagttagtgatgatattgcggaaatcccaatgataacttattctccgcaacctattgctaactttattgtcctgttcaccagtgggagtacaggcaaaccaaaagccattagtatttcagaatcgttagtatgtcgtcgaatttattcggtgaccgagaaattaaaatttacgcaagatgccaaaatattcatgtcaggtttgttgaataatacaactggagtgattttttctttcggctcattattgcatcaatcaacactttttatacccgaagatagaaatgtagagagatggcctgattatctttctcgcaataaaatcactcatattatgttacgcccagaatcaatgaaattattcgttaaatcgacagcagaacttaatattgatctctcttgtttacgggtggttgcttatggcgctgcggcgatgcctcctagcgtacttgagaaagggcgacaattaattggctgtgaatgggtgcagggatatgggttaagtgaaacttatggtcctttctgttgggtggatgagcaagatcatcgtgataaaagatatctcaattcaatttattgtgttggtaagattgataatacattggaagtggcagttaaacctattataggttcatcggataatatcggagaaattatactaaggggtaaaagtattatggaaggatattatgatgtcctttctggagaaataacgcctcctgatgaatggtttgccactggtgatcttggttatatagatgaagagggttatttagttttgaaaggacgtaagcaaaatacgtttatgagtgctaacggacacagaatttatcctgaagaaattgaatctattttatcccgaatacccaatgtgaatgtcgctacggttgttggtttttctttccatgaaaatggtgttgctattgatcagccggttgcttgcatgagtggagagatatctaagaagtcattacctgaaattgaagatattatttcatcatttttaatgagtaaactcagtcgagaaaaatggccggattggttctatgttactgatgaatgctttccgaaaagccataatgataagatattgaaatcagagttaattaaatcaatcgatcctaagaaattatttacattgaggaaccaataaaaggagatataccatggacggtatttctttatcatctgatttttttgatctttggattatcaaaatcgacgatattgatttagcttctattgaacagttaattcactgttctgatatagttcgccataaccaaatttgtttagcggatagaagaaagagatttatatttagacgggctgcattacgttatgttttgagtcaatatttatctgattatgaaatcataacgaatgataacggaaaaccttatatatccacggagcaagacttcaaatattatttttcactgagtgcttcaggaaactattgtgccattggttttagctcaagggaaataggtgttgatattgaagtcactccttctaaggtaaaattttcagaaattattgaacgttttattaaggataaagatttggaatatatgaaaggtataatgttaaaacaactatcaggagttagtctcggatttaataactattatcatttaatgtcattatattattgggttagacttgaagcatatattaaattatttgcttcgactttacatgagaaattattggttaataactctgattctgttaaagatatgaaagaattggaggcaagcacattattgattcatagtcagcaatttgtttgtgccttatctcaaaagaaagtcatttctacaccaaatatcaaggaaataaattattccgaaattataaggaacaaagatgagtaatcgaacagaaagtaatcgtattgtacacggccgcataatcgaaattaatacgactcactataggggaattgtgagcggataacaattccccatcttagtatattagttaagtataagaaggagatatacatatgcgtcatgtagagcatacagtcaccgttgcggccccagcagacttggtttgggaggtacttgccgatgtcttaggctatgctgacatcttcccaccgacggaaaaagttgaaattcttgaggaggggcaaggataccaggtagtgcgccttcacgtcgatgttgcgggtgagattaatacatggaccagtcgtcgcgatttagaccctgcgcgccgcgtaattgcttaccgccaacttgagacggctccgatcgtgggccacatgagcggggaatggcgtgctttcacactggatgccgaacgtacccaattagtcctgactcacgatttcgtaacccgtgcagccggggatgacggtttagtcgccggaaaattgaccccagatgaggcgcgcgaaatgttagaagcggtggtagaacgtaactctgtcgccgacttaaacgcggtccttggagaagctgagcgtcgcgtccgcgcagccggtggagttggtaccgtaactgcgtaataataattttgtttaactttaagaaggagatatatccatgtcagggcgcaaaacctttttagacttaagttttgctacccgcgacacaccgtcggaggcgactccggtggtggtagatttgctggaccacgtaactggagccaccgtattaggattatcacctgaggatttccccgatggtatggctatttccaatgagaccgttacgttgacgacccacactggcacgcacatggatgcgccactgcactatggtcccttaagtgggggagttccggcaaagtcgattgaccaagtgcccttggaatggtgctatggacctggagttcgtttggatgttcgccacgtgccggcaggagatggtattactgtcgatcatttgaacgccgcgttggatgcagcagagcacgatttggcccccggtgacattgtgatgctgtggaccggcgcggacgctctgtggggaacccgcgaatacttgagcacgtttccggggttaactgggaaggggacacaatttttggtcgaggcgggtgttaaagtcattggcattgatgcatggggactggatcgcccgatggcagctatgatcgaagaataccgtcgtacgggcgataaaggagcattatggccggctcacgtctatggacgcacacgcgaatacctgcaattagagaagcttaataatttgggcgctttaccaggagctacagggtatgacatttcatgctttccggttgcggttgcaggcactggagctgggtggactcgtgtggtcgccgttttcgagcaagaggaagaggattaa

SEQ ID NO: 5 - DNA, 1470 bp, Escherichia coli
  source 1..1470: mol_type = other DNA
atggcactgacgaaaccggatgttgatgttctggttgttggtggtggtctgggtggtctgagcacggcgctgtttctggcgcgccgcggtgcacgtgttctgctggttgaacgtcatgcgagtactagcgttctgccgaaagcggcgggtcagaatccgcgtacaatggaactgtttcgttttggtggtgttgcagatgaaattctggcaaccgatgatattcgtggtgcacagggtgattttacaattaaagttgttgaacgtgtgggtggtcgtgttccggcgcagctgcgtgaatcttttgaagaactggtgggtgcaacagaacagtgtacaccgatgccgtgggccctggcaccgcaggatcgcgttgaaccggttctggttgcacatgcagcaaaacatggtgctgaaattcgttttgcaactgaactgacctcatttcaggcgggcgatgatggtgttaccgcacgtctgcgtgatctgggtaccggtgccgaaagtacagtcagcgcacgctatctggttgcagcagatggtccgcgtagcgccattcgtgaaagcctgggtattacccgtcatggccacggtaccctggcacattttatgggtgttatttttgaagcagatctgaccgccgttgttccgaaaggtagtacaggttggtattatctgcagcatcctgattttaccggtacctttggtccgaccgatcgtccgaatcgtcataccttttatgttcgttatgatccggaacgtggtgaacgtccggaagattatacgccgcagcgttgtaccgaactgattcgcctggcagttgatgcaccgggtctggttccggatattctggatattcaggcatgggatatggcggcttatattgcagatcgttggcgtgaaggtccggttctgctggtgggtgatgcagcaaaagttaccccgcctacaggtggtatgggtggtaataccgcaattggtcatggttttgatgttgcgtggaaactggccgcggttctgcgtggtgaagccggtgaacgtctgctggatagctatggtgccgatggcagtctggttagtcgtctggttgttgatgaaagcctggcgatttatgcacagcgtatggcaccgcatctgctgggtagtgttccggaagaacgtggtaccgcacaggttgttctgggttttcgttatcgttctactgcagttgcagcagaagatgatgatccggaaccgactgaagatcctcgtcgtcctagtggtcgtcctggttttcgtgcaccgcatgtttggattgaacaggatggtacccgtcgtagcacagtggaactgtttggtgattgttgggtgctgctggcagcgccggaaggtggtgcatggggtcaggcagctgctcgtgctgcacgtatttgggcaagtgcaagtacatctattagcagcgcggcaatgagtccgccgccgcctgctaattaa

SEQ ID NO: 6 - DNA, 1434 bp, Escherichia coli
  source 1..1434: mol_type = other DNA
atgggcagcctgaccaacaacgataacctgcatatttttctggtttgttttatcggtcagggtgttgttaacccgatgctgcgtctgggtaaagcatttgcaagcaaaggtctgctggttacgctgtctgcaccggaaattgtgggtactgaaattcgtaaagccaataacctgaatgatgatcagccgattaaagttggtagcggtatgattcgttttgaattttttgatgatggctgggaatctgttaatggtagcaaaccgtttgatgtttggcagtatatcaaccatctggatcagacaggtcgtcagaaactgccaattatgctgaaaaagcatgaagaaacgggtacgccggtgagctgtctgattctgaatcctctggtgccgtgggttgccgatgtggcggatagtctgcagattccttgtgccaccctgtgggtacagagctgtgcaagctttagcgcatattatcattatcatcatggtctggttccgtttccgaccgaaagtgaaccggaaattgatgttcagctgccgggtatgccgctgctgaaatatgatgaagttccggattttctgcatcctcgtaccccgtatccgttttttggtaccaatattctgggtcagtttaaaaatctgtcaaaaaatttttgtattctgatggataccttttatgaactggaacatgaaattattgataatatgtgtaaactgtgtccgattaaaccgattggtccgctgtttaaaattccgaaagatccgagcagcaatggtattaccggtaattttatgaaagttgatgattgtaaagaatggctggatagccgtccgaccagcaccgttgtttatgttagcgttggtagcgttgtttatctgaaacaggaacaggttaccgaaatggcatacggtattctgaatagcgaagttagctttctgtgggttctgcgtccgccgagcaaacgtattggtaccgaaccgcatgttctgccggaagaattttgggaaaaagcaggtgatcgtggtaaagttgttcagtggagcccgcaggaacaggttctggcacatccggcaaccgttggttttctgacccattgtggttggaatagcacccaggaagcaattagcagcggtgttccggttattacctttccgcagtttggtgatcaggttaccaatgcaaaatttctggttgaagaatttaaagttggtgttcgtctgggtcgtggtgaactggaaaatcgtattattacccgtgatgaagttgaacgtgcactgcgtgaaattaccagcggtccgaaagcagaagaagttaaagaaaatgcactgaaatggaaaaaaaaagcagaagaaaccgttgcaaaaggtggttatagcgaacgtaatctggttggttttattgaagaagttgcacgtaaaaccggtaccaaataa

(19) United States
(12) Patent Application Publication
(10) Pub. No.: US 2025/0215471 A1
(43) Pub. Date: Jul. 3, 2025

(54) GENETICALLY MODIFIED MICROORGANISM, PREPARATION METHOD THEREOF, AND METHOD OF PRODUCING TARGET CHEMICAL

(71) Applicant: INDUSTRIAL TECHNOLOGY RESEARCH INSTITUTE, Hsinchu (TW)

(72) Inventors: Pei-Ching CHANG, Zhubei City (TW); Hung-Yu LIAO, Taoyuan City (TW); Jhong-De LIN, Hsinchu City (TW); Jie-Len HUANG, Hsinchu City (TW); Ke-Ming LIANG, Hsinchu City (TW)

(73) Assignee: INDUSTRIAL TECHNOLOGY RESEARCH INSTITUTE, Hsinchu (TW)

(21) Appl. No.: 18/400,179

(22) Filed: Dec. 29, 2023

(51) Int. Cl. C12P 19/00; C12N 1/20; C12N 9/00; C12N 9/02; C12N 9/10; C12N 9/88; C12R 1/19

(52) U.S. Cl. CPC C12P 19/00; C12N 1/20; C12N 9/0073; C12N 9/1029; C12N 9/1051; C12N 9/88; C12N 9/93; C12N 2510/00; C12R 2001/19; C12Y 114/13; C12Y 114/14; C12Y 203/01; C12Y 204/01; C12Y 406/00; C12Y 602/01001

(57) ABSTRACT

A genetically engineered microorganism is provided. The genetically engineered microorganism has a higher expression level of acid-tolerant gene than a source microorganism. The acid-tolerant gene includes at least one of dsdA, dcuC and glaA. A method of preparing the genetically engineered microorganism and a method of producing a target chemical using the genetically engineered microorganism are also provided.

### INCORPORATION BY REFERENCE OF SEQUENCE LISTING

A sequence listing submitted as an ST.26 XML format is incorporated herein by reference. The file containing the sequence listing is named “9044C-P230228000-US_Seq”; its date of creation was Dec. 28, 2023; and its size is 19 kilobytes.

### TECHNICAL FIELD

The present invention relates to a genetically engineered microorganism, a preparation method thereof, and a method of producing a target chemical.

### BACKGROUND

Chemical synthesis is often highly dependent on petrochemical feedstocks and has high carbon emissions. Under the global trend of reducing carbon emissions and achieving net zero carbon emissions, biosynthetic technology is gradually receiving attention because it is in line with future environmental protection trends.

Take “carmine”, a dye widely used in high-priced cosmetics, as an example. Because it contains a complex structure in which an anthraquinone polyketide is connected to a glucose molecule, it is very difficult to prepare using chemical synthesis, and the yield is not high. Furthermore, the high-temperature and high-pressure reaction conditions and the use of harmful substances in the manufacturing process are detrimental to the environment. Therefore, the current method of producing carmine mainly uses cochineals for extraction. However, cochineal has a long growth cycle, and is subject to geographical and climatic restrictions, resulting in unstable output and quality, and the extraction steps thereof are complicated, making it difficult to mass-produce. In addition, low-carbon, non-animal and cruelty-free products have gradually entered the mainstream of today's brand appeal.

Accordingly, developing a biosynthetic method that can improve the production efficiency of carmine, thereby improving the market competitiveness of carmine, has become one of the current issues that the industry is eager to solve.

### SUMMARY

In accordance with some embodiments of the present disclosure, a genetically engineered microorganism is provided, which includes a higher expression level of acid-tolerant genes compared to a source microorganism. The acid-tolerant gene includes at least one of dsdA, dcuC and glaA.

In accordance with some embodiments of the present disclosure, the aforementioned genetically engineered microorganism further includes at least one exogenous nucleotide sequence encoding acetyl-CoA synthetase (ACS).

In accordance with other embodiments of the present disclosure, a novel genetically engineered E. coli strain is provided, and its deposit number is BCRC 940699. The novel genetically engineered E. coli strain has a higher expression level of acid-tolerant gene compared to a source microorganism, and the acid-tolerant gene includes at least one of dsdA, dcuC and glaA. In addition, the novel genetically engineered E. coli strain includes exogenous nucleotide sequences encoding acetyl-CoA synthetase and encoding an enzyme related to carmine synthesis.

In accordance with other embodiments of the present disclosure, a method of preparing a genetically engineered microorganism is provided. The method includes the following steps: (a) acclimating a microorganism with an acidic culture solution; (b) introducing an exogenous nucleotide sequence encoding acetyl-CoA synthetase into the acclimated microorganisms; and (c) introducing an exogenous nucleotide sequence encoding an enzyme related to the synthesis of a target chemical into the acclimated microorganism, to obtain the genetically engineered microorganism.

In accordance with further embodiments of the present disclosure, a method of producing a target chemical is provided. The method includes the following steps: (a) providing the aforementioned genetically engineered microorganism; (b) culturing the genetically engineered microorganism in an acidic culture solution; (c) culturing the genetically engineered microorganism at 15° C. to 42° C. for 8 hours to 80 hours to produce a culture solution containing the target chemical; and (d) isolating and purifying the target chemical from the culture solution in step (c).

In accordance with some embodiments of the present disclosure, the aforementioned target chemical includes carmine.

### BRIEF DESCRIPTION OF THE DRAWINGS

The present invention can be more fully understood by reading the subsequent detailed description and examples with references made to the accompanying drawings, wherein:

FIG. 1 shows a biosynthetic pathway diagram of carmine constructed by genetically engineered microorganisms in accordance with some embodiments of the present disclosure;

FIG. 2A to FIG. 2C show the growth conditions of acclimated acid-tolerant strains in culture media of different pH values in accordance with some embodiments of the present disclosure; FIG. 2A shows the growth condition of acclimated acid-tolerant strains in a medium of pH 7. FIG. 2B shows the growth condition of the aforementioned acclimated acid-tolerant strains cultured in a medium of pH 4 for 24 hours; and FIG. 2C shows the growth condition of the aforementioned acclimated acid-tolerant strains once again cultured in a medium of pH 4;

FIG. 3 shows the gene expression analysis result of acclimated acid-tolerant strains analyzed by next-generation sequencing (NGS) in accordance with some embodiments of the present disclosure. The red dots in the figure represent genes with up-regulated expression (relative to native E. coli), the green dots represent genes with down-regulated expression (relative to native E. coli), and the blue dots represent no change in expression;

FIG. 4 shows a plasmid construction map of an acetic acid metabolism-enhanced acid-tolerant strain in accordance with some embodiments of the present disclosure;

FIG. 5A and FIG. 5B show the growth conditions of native strains, acid-tolerant strains and acetic acid metabolism-enhanced acid-tolerant strains in different culture media in accordance with some embodiments of the present disclosure. FIG. 5A shows the analysis results of strain growth density of native strains, acid-tolerant strains and acetic acid metabolism-enhanced acid-tolerant strains grown in hydrochloric acid basal culture solution for 0 hours, 24 hours and 48 hours; and FIG. 5B shows the analysis results of strain growth density of native strains, acid-tolerant strains and acetic acid metabolism-enhanced acid-tolerant strains grown in acetic acid basal culture solution for 0 hours, 24 hours and 48 hours;

FIG. 6 shows a plasmid construction map of a carmine-producing strain in accordance with some embodiments of the present disclosure;

FIG. 7 shows a plasmid construction map of a carmine-producing strain in accordance with some embodiments of the present disclosure;

FIG. 8A and FIG. 8B show the carmine production condition of acid-tolerant strains and acetic acid metabolism-enhanced acid-tolerant strains used for carmine production in different culture media in accordance with some embodiments of the present disclosure. FIG. 8A shows the analysis results of carmine production of acid-tolerant strains and acetic acid metabolism-enhanced acid-tolerant strains used for carmine production in hydrochloric acid basal culture solution for 0 hours and 48 hours; and FIG. 8B shows the analysis results of carmine production of acid-tolerant strains and acetic acid metabolism-enhanced acid-tolerant strains used for carmine production in acetic acid basal culture solution for 0 hours and 48 hours.

### DETAILED DESCRIPTION

The genetically engineered microorganism, the preparation method thereof, and the method of producing a target chemical of the present disclosure are described in detail in the following description. It should be understood that in the following detailed description, for purposes of explanation, numerous specific details and embodiments are set forth in order to provide a thorough understanding of the present disclosure. The specific elements and configurations described in the following detailed description are set forth in order to clearly describe the present disclosure. It will be apparent that the exemplary embodiments set forth herein are used merely for the purpose of illustration and not the limitations of the present disclosure.

Unless defined otherwise, all technical and scientific terms used herein have the same meaning as commonly understood by one of ordinary skill in the art to which this disclosure belongs. It should be appreciated that, in each case, the term, which is defined in a commonly used dictionary, should be interpreted as having a meaning that conforms to the relative skills of the present disclosure and the background or the context of the present disclosure, and should not be interpreted in an idealized or overly formal manner unless so defined.

In the biological world, the main anabolic raw materials of carmine are acetyl coenzyme A (acetyl-CoA) and malonyl coenzyme A (malonyl-CoA), which can be catalyzed by polyketide synthase (PKS) for a condensation reaction to synthesize carmine. Increasing the production of metabolic raw materials can provide sufficient precursors for subsequent reactions, allowing microorganisms to effectively biosynthesize carmine. However, microorganisms can easily direct metabolic resources to acetic acid production under adverse conditions, thereby affecting their own growth and carmine production.

Based on this key physiological mechanism, the embodiments of the present disclosure screen and establish strains that are tolerant to acidic environments and can utilize acetic acid as a carbon source, and thereby further prepare genetically engineered strains that can produce a target chemical (for example, carmine). By improving the strain's ability to convert acetic acid into metabolic raw materials for the target chemical, the acetic acid utilization rate of the strains and the yield of the target chemical can be improved. Furthermore, the genetically engineered strains of the embodiments of the present disclosure also increase the possibility of industrial production of anthraquinone compounds related to the biosynthesis of carmine.

In accordance with the embodiments of the present disclosure, a genetically engineered microorganism is provided, which has a higher expression level of acid-tolerant genes compared with a source microorganism, and the acid-tolerant genes include at least one of dsdA, dcuC and glaA. In other words, compared with the source microorganism, the expression level of at least one of the dsdA, dcuC and glaA genes of the genetically engineered microorganism is higher. In accordance with some embodiments, compared with the source microorganism, the expression level of the dsdA gene of the genetically engineered microorganism of the present disclosure is increased by at least about 5 times to about 20 times, or about 7 times to about 18 times, or about 9 times to about 16 times, for example, about 12.61 times; the expression level of the dcuC gene is increased by at least about 3 times to about 18 times, or about 5 times to about 16 times, for example, about 11.15 times; and the expression level of the glaA gene is increased by at least about 2 times to about 16 times, or about 4 times to about 14 times, for example, about 10.38 times.

In accordance with some embodiments, compared to the source microorganism, the expression level of at least one of the yhjx, recA, FliA, and NifE genes of the genetically engineered microorganism of the present disclosure is also higher. In accordance with some embodiments, compared with the source microorganism, the expression level of the yhjx gene of the genetically engineered microorganism of the present disclosure is increased by at least about 2 times to about 15 times, or about 4 times to about 13 times, for example, about 9.41 times; the expression level of recA gene is increased by at least about 3 times to about 18 times, or about 5 times to about 16 times, for example, about 11.02 times; the expression level of the FliA gene is increased by at least about 3 times to about 18 times, or about 5 times to about 16 times, for example, about 11.11 times, and the expression level of the NifE gene is increased by at least about 3 times to about 18 times, or about 5 times to about 16 times, for example, about 11.68 times.

In accordance with some embodiments, the genetically engineered microorganism of the present disclosure has tolerance to an acidic environment. The pH value of the aforementioned acidic environment can range from pH 3 to pH 5, for example, pH 3.2, pH 3.5, pH 3.8, pH 4, pH 4.2, pH 4.5 or pH 4.8, but it is not limited thereto. In accordance with some embodiments, the genetically engineered microorganism of the present disclosure can survive in an acidic environment of pH 3 to pH 5, and the survival rate after 24 hours of culture can reach more than 90%, for example, up to 95%, 96%, 97%, 98%, 99% or 100%, but it is not limited thereto.

In accordance with some embodiments, the source microorganism of the genetically engineered microorganism of the present disclosure may include Escherichia coli, Corynebacterium glutamicum, Bacillus subtilis, Pseudomonas putida, Yarrowia lipolytica, Saccharomyces cerevisiae or Pichia pastoris, but it is not limited thereto. In accordance with some embodiments, the aforementioned E. coli may include E. coli K12, BW25113, DH5a, BL21, XL1-blue or other suitable strains, but it is not limited thereto.

In accordance with some embodiments, the genetically engineered microorganism of the present disclosure may further include at least one exogenous nucleotide sequence encoding acetyl-CoA synthetase (ACS). The exogenous nucleotide sequence encoding acetyl-CoA synthetase may include a sequence having at least 85% sequence similarity with SEQ ID NO: 1, for example, may include a sequence having at least 88%, 90%, 92%, 95%, 98% or 99% sequence similarity with SEQ ID NO: 1, but it is not limited thereto. In accordance with some embodiments, the aforementioned exogenous nucleotide sequence encoding acetyl-CoA synthetase may include the sequence of SEQ ID NO: 1. In accordance with some embodiments, the aforementioned exogenous nucleotide sequence encoding acetyl-CoA synthetase may be the sequence of SEQ ID NO: 1.

In accordance with some embodiments, the genetically engineered microorganism of the present disclosure may further include an exogenous nucleotide sequence encoding an enzyme related to the synthesis of a target chemical. In accordance with some embodiments, the aforementioned target chemical may include carmine, para-aminobenzoic acid (PABA), indigo blue, melanin, or a combination thereof. In accordance with some embodiments, the aforementioned target chemical is carmine, and the enzymes related to the synthesis of carmine may include at least one of the following: cyclase ZhuI, aromatase ZhuJ, polyketide synthase (octaketide synthase complex antDEFBG), hydroxylase dnrFP217K, glucosyltransferase GtCGTV93Q/Y193F, glucosyltransferase UGT2, monooxygenase aptC and 4′-phosphopantetheinyl transferase npgA, but it is not limited thereto. In accordance with some embodiments, the enzyme related to the synthesis of the target chemical may include glutamine aminotransferase (aminodeoxychorismate synthase component 2) PabA, 4-amino-4-deoxychorismate synthase (aminodeoxychorismate synthase component 2) PabB, 4-amino-4-deoxychorismate lyase (aminodeoxychorismate lyase) PabC, naphthalene dioxygenase NDO or a combination thereof, but it is not limited thereto.

In accordance with some embodiments, the aforementioned exogenous nucleotide sequence encoding cyclase ZhuI may include a sequence having at least 85% sequence similarity with SEQ ID NO: 2, for example, may include a sequence having at least 88%, 90%, 92%, 95%, 98% or 99% sequence similarity with SEQ ID NO: 2, but it is not limited thereto. In accordance with some embodiments, the aforementioned exogenous nucleotide sequence encoding cyclase ZhuI may include the sequence of SEQ ID NO: 2. In accordance with some embodiments, the aforementioned exogenous nucleotide sequence encoding cyclase ZhuI may be the sequence of SEQ ID NO: 2.

In accordance with some embodiments, the aforementioned exogenous nucleotide sequence encoding aromatase ZhuJ may include a sequence with at least 85% sequence similarity with SEQ ID NO:3, for example, may include a sequence having at least 88%, 90%, 92%, 95%, 98% or 99% sequence similarity with SEQ ID NO:3, but it is not limited thereto. In accordance with some embodiments, the aforementioned exogenous nucleotide sequence encoding aromatase ZhuJ may include the sequence of SEQ ID NO: 3. In accordance with some embodiments, the aforementioned exogenous nucleotide sequence encoding aromatase ZhuJ may be the sequence of SEQ ID NO: 3.

In accordance with some embodiments, the aforementioned exogenous nucleotide sequence encoding octaketide synthase complex antDEFBG may include a sequence with at least 85% sequence similarity with SEQ ID NO: 4, for example, may include a sequence having at least 88%, 90%, 92%, 95%, 98% or 99% sequence similarity with SEQ ID NO: 4, but it is not limited thereto. In accordance with some embodiments, the aforementioned exogenous nucleotide sequence encoding octaketide synthase complex antDEFBG may include the sequence of SEQ ID NO: 4. In accordance with some embodiments, the aforementioned exogenous nucleotide sequence encoding octaketide synthase complex antDEFBG may be the sequence of SEQ ID NO: 4.

In accordance with some embodiments, the aforementioned exogenous nucleotide sequence encoding hydroxylase dnrFP217K may include a sequence with at least 85% sequence similarity with SEQ ID NO: 5, for example, may include a sequence having at least 88%, 90%, 92%, 95%, 98% or 99% sequence similarity with SEQ ID NO: 5, but it is not limited thereto. In accordance with some embodiments, the aforementioned exogenous nucleotide sequence encoding hydroxylase dnrFP217K may include the sequence of SEQ ID NO: 5. In accordance with some embodiments, the aforementioned exogenous nucleotide sequence encoding hydroxylase dnrFP217K may be the sequence of SEQ ID NO: 5.

In accordance with some embodiments, the aforementioned exogenous nucleotide sequence encoding glycosyltransferase GtCGTV93Q/Y193F may include a sequence with at least 85% sequence similarity with SEQ ID NO: 6, for example, may include a sequence having at least 88%, 90%, 92%, 95%, 98% or 99% sequence similarity with SEQ ID NO: 6, but it is not limited thereto. In accordance with some embodiments, the aforementioned exogenous nucleotide sequence encoding glycosyltransferase GtCGTV93Q/Y193F may include the sequence of SEQ ID NO: 6. In accordance with some embodiments, the aforementioned exogenous nucleotide sequence encoding the glycosyltransferase GtCGTV93Q/Y193F may be the sequence of SEQ ID NO: 6.

In accordance with some embodiments, the aforementioned genetically engineered microorganism including an exogenous nucleotide sequence encoding acetyl-CoA synthase and an exogenous nucleotide sequence encoding an enzyme related to the synthesis of carmine has a deposit number of BCRC 940699.

Furthermore, in accordance with some embodiments of the present disclosure, a novel genetically engineered E. coli strain is provided, and its deposit number is BCRC 940699. The aforementioned novel genetically engineered E. coli strain has a higher expression level of acid-tolerant genes than the source microorganism. The aforementioned acid-tolerant genes may include at least one of dsdA, dcuC and glaA, and the aforementioned novel genetically engineered E. coli strain may include exogenous nucleotide sequences encoding acetyl-CoA synthetase and encoding an enzyme related to the synthesis of carmine.

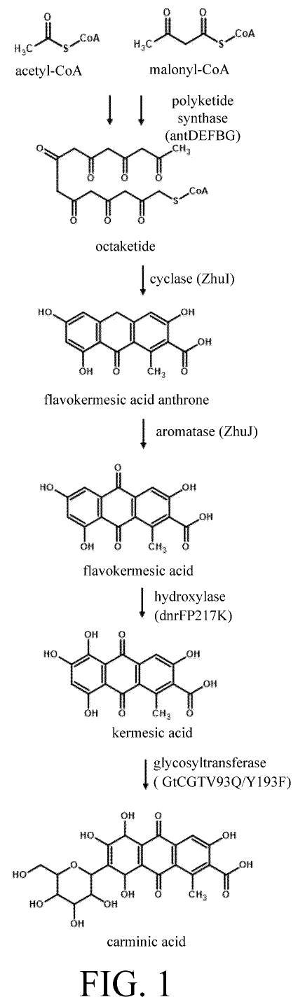

Please refer to FIG. 1. FIG. 1 shows a biosynthetic pathway diagram of carmine constructed by genetically engineered microorganisms in accordance with some embodiments of the present disclosure. As shown in FIG. 1, the genetically engineered microorganism of the present disclosure can use acetic acid and/or its salts to generate acetyl-CoA and further accumulate malonyl-CoA, and then the acetyl-CoA and malonyl-CoA are condensed to synthesize octaketide through polyketide synthase (such as octaketide synthase complex antDEFBG). Octaketide is converted into flavokermesic acid anthrone (FKA) through the action of cyclase (such as ZhuI). Flavokermesic acid anthrone is then converted into flavokermesic acid through the action of aromatase (such as ZhuJ). Flavokermesic acid is then converted into kermesic acid through the action of hydroxylase (such as dnrFP217K). Finally, kermesic acid is converted into carminic acid through glycosyltransferase (such as GtCGTV93Q/Y193F). Carminic acid is then added with aluminum salt or calcium salt to obtain carmine.

It should be noted that in accordance with the embodiments of the present disclosure, the genetically engineered microorganism is tolerant to an acidic environment and can utilize relatively low-price acetic acid and/or its salts as a carbon source to grow and synthesize carmine. Therefore, the production cost can be effectively reduced and the output of carmine can be increased.

In addition, in accordance with the embodiments of the present disclosure, a method of producing a target chemical is also provided, which includes step (a) providing the aforementioned genetically engineered microorganism; and step (b) culturing the genetically engineered microorganism in an acidic culture solution.

In accordance with some embodiments, the pH value of the acidic culture solution ranges from pH 3 to pH 5, for example, pH 3.2, pH 3.5, pH 3.8, pH 4, pH 4.2, pH 4.5 or pH 4.8, but it is not limited thereto. In accordance with some embodiments, the acidic culture solution may include acetic acid and/or its salts, but it is not limited thereto. The genetically engineered microorganisms of the present disclosure can use acetic acid and/or its salts as carbon sources for growth.

Furthermore, the aforementioned method of producing a target chemical may include step (c) of culturing the genetically engineered microorganisms at the temperature of 15° C. to 42° C. for 8 hours to 80 hours to produce a culture solution containing the target chemical.

In accordance with some embodiments, the temperature for culturing the genetically engineered microorganism to produce the target chemical may be ranging from 20° C. to 40° C., such as 21° C., 22° C., 23° C., 24° C., 25° C., 26° C., 27° C., 28° C., 29° C., 30° C., 31° C., 32° C., 33° C., 34° C., 35° C., 36° C., 37° C., 38° C. or 39° C., but it is not limited thereto. Furthermore, in accordance with some embodiments, the time for culturing the genetically engineered microorganism to produce the target chemical may be ranging from 10 hours to 75 hours, or from 15 hours to 70 hours, from 20 hours to 65 hours, from 25 hours to 60 hours, from 30 hours to 55 hours, from 35 hours to 50 hours, or from 40 hours to 45 hours, but it is not limited thereto.

Moreover, the aforementioned method of producing a target chemical may include step (d) of isolating and purifying the target chemical from the culture solution of step (c).

The aforementioned target chemical can be isolated and purified from the culture solution by any suitable method. In accordance with some embodiments, the target chemical may include carmine, but it is not limited thereto.

In addition, in accordance with the embodiments of the present disclosure, a method of preparing a genetically engineered microorganism is further provided, which includes step (a) acclimating a microorganism (i.e. source microorganism) through an acidic culture solution.

In accordance with some embodiments, the aforementioned source microorganism for preparing the genetically engineered microorganism may include Escherichia coli, Corynebacterium glutamicum, Bacillus subtilis, Pseudomonas putida, Yarrowia lipolytica, Saccharomyces cerevisiae or Pichia pastoris, but it is not limited thereto. In accordance with some embodiments, the aforementioned E. coli may include E. coli K12, BW25113, DH5a, BL21, XL1-blue or other suitable strains, but it is not limited thereto.

In accordance with some embodiments, the step of acclimation may include preparing a plurality of acidic culture solutions with different pH values ranging from pH 6.5 to pH 4 and from the acidic culture solutions, culturing the microorganism sequentially with the culture solution with a high pH value to a low pH value. In accordance with some embodiments, the pH values of the plurality of acidic culture solutions include three or more of pH 6.5, pH 6, pH 5.5, pH 5, pH 4.5 and pH 4.

In accordance with some embodiments, the acidic culture solution used for culturing the microorganism for acclimation may include acetic acid, hydrochloric acid, citric acid, salts of the aforementioned acids, a combination thereof, or other suitable acids, but it is not limited thereto. In accordance with some embodiments, the substrate of the culture solution may include LB medium, YNB medium, YPD medium or other suitable medium, but it is not limited thereto. Specifically, the aforementioned acetic acid, hydrochloric acid, citric acid, salts of the aforementioned acids, or a combination thereof can be added to the substrate of the culture solution to adjust the pH value of the culture solution to be ranging from pH 6.5 to pH 4.

In accordance with some embodiments, a plurality of acidic culture solutions with different pH values can be used to culture the microorganism for 10 days to 20 days respectively, for example, 11 days, 12 days, 13 days, 14 days, 15 days, 16 days, 17 days, 18 days or 19 days, but it is not limited thereto. In accordance with some embodiments, the temperature for culturing the acclimated microorganism may be ranging from 25° C. to 37° C., for example, 26° C., 27° C., 28° C., 29° C., 30° C., 31° C., 32° C., 33° C., 34° C., 35° C. or 36° C., but it is not limited thereto.

For example, in accordance with some embodiments, acidic culture solutions of pH 6.5, pH 6, pH 5.5, pH 5, pH 4.5 and pH 4 can be prepared, and the acidic culture solutions of pH 6.5, pH 6, pH 5.5, pH 5, pH 4.5 and pH 4 may be sequentially used to culture the microorganism at 30° C. for 14 days, to obtain the acclimated microorganism that is tolerant to an acidic environment. In accordance with some embodiments, the aforementioned acclimated microorganism can survive in an acidic environment of pH 3 to pH 5, for example, the survival rate of the acclimated microorganism after 24 hours of culture can reach more than 90%, such as 95%, 96%, 97%, 98%, 99% or 100%.

Furthermore, the aforementioned method of preparing a genetically engineered microorganism may include step (b) of introducing an exogenous nucleotide sequence encoding acetyl-CoA synthetase into the acclimated microorganism.

In accordance with some embodiments, the aforementioned exogenous nucleotide sequence encoding acetyl-CoA synthase may include a sequence with at least 85% sequence similarity with SEQ ID NO: 1, for example, may include a sequence having at least 88%, 90%, 92%, 95%, 98% or 99% sequence similarity with SEQ ID NO: 1, but it is not limited thereto. In accordance with some embodiments, the aforementioned exogenous nucleotide sequence encoding acetyl-CoA synthetase may include the sequence of SEQ ID NO: 1. In accordance with some embodiments, the aforementioned exogenous nucleotide sequence encoding acetyl-CoA synthetase may be the sequence of SEQ ID NO: 1.

Furthermore, the aforementioned method of preparing a genetically engineered microorganism may include step (c) of introducing an exogenous nucleotide sequence encoding an enzyme related to the synthesis of the target chemical into the acclimated microorganism to obtain the genetically engineered microorganism.

In accordance with some embodiments, the aforementioned target chemical may include carmine, p-aminobenzoic acid, indigo blue, melanin or a combination thereof. In accordance with some embodiments, the aforementioned target chemical is carmine, and the enzyme related to the synthesis of carmine may include at least one of the following: cyclase ZhuI, aromatase ZhuJ, polyketide synthase (octaketide synthase complex antDEFBG), hydroxylase dnrFP217K, glucosyltransferase GtCGTV93Q/Y193F, glucosyltransferase UGT2, monooxygenase aptC and 4′-phosphopantetheinyl transferase npgA, but it is not limited thereto. In accordance with some embodiments, the enzyme related to the synthesis of the target chemical may include glutamine aminotransferase (aminodeoxychorismate synthase component 2) PabA, 4-amino-4-deoxychorismate synthase (aminodeoxychorismate synthase component 2) PabB, 4-amino-4-deoxychorismate lyase (aminodeoxychorismate lyase) PabC, naphthalene dioxygenase NDO or a combination thereof, but it is not limited thereto.

In accordance with some embodiments, the aforementioned exogenous nucleotide sequence encoding cyclase ZhuI may include a sequence having at least 85% sequence similarity with SEQ ID NO: 2, for example, may include a sequence having at least 88%, 90%, 92%, 95%, 98% or 99% sequence similarity with SEQ ID NO: 2, but it is not limited thereto. In accordance with some embodiments, the aforementioned exogenous nucleotide sequence encoding cyclase ZhuI may include the sequence of SEQ ID NO: 2. In accordance with some embodiments, the aforementioned exogenous nucleotide sequence encoding cyclase ZhuI may be the sequence of SEQ ID NO: 2.

In accordance with some embodiments, the aforementioned exogenous nucleotide sequence encoding aromatase ZhuJ may include a sequence with at least 85% sequence similarity with SEQ ID NO: 3, for example, may include a sequence having at least 88%, 90%, 92%, 95%, 98% or 99% sequence similarity with SEQ ID NO: 3, but it is not limited thereto. In accordance with some embodiments, the aforementioned exogenous nucleotide sequence encoding aromatase ZhuJ may include the sequence of SEQ ID NO: 3. In accordance with some embodiments, the aforementioned exogenous nucleotide sequence encoding aromatase ZhuJ may be the sequence of SEQ ID NO: 3.

In accordance with some embodiments, the aforementioned exogenous nucleotide sequence encoding octaketide synthase complex antDEFBG may include a sequence with at least 85% sequence similarity with SEQ ID NO: 4, for example, may include a sequence having at least 88%, 90%, 92%, 95%, 98% or 99% sequence similarity with SEQ ID NO: 4, but it is not limited thereto. In accordance with some embodiments, the aforementioned exogenous nucleotide sequence encoding octaketide synthase complex antDEFBG may include the sequence of SEQ ID NO: 4. In accordance with some embodiments, the aforementioned exogenous nucleotide sequence encoding octaketide synthase complex antDEFBG may be the sequence of SEQ ID NO: 4.

In accordance with some embodiments, the aforementioned exogenous nucleotide sequence encoding hydroxylase dnrFP217K may include a sequence with at least 85% sequence similarity with SEQ ID NO: 5, for example, may include a sequence having at least 88%, 90%, 92%, 95%, 98% or 99% sequence similarity with SEQ ID NO: 5, but it is not limited thereto. In accordance with some embodiments, the aforementioned exogenous nucleotide sequence encoding hydroxylase dnrFP217K may include the sequence of SEQ ID NO: 5. In accordance with some embodiments, the aforementioned exogenous nucleotide sequence encoding hydroxylase dnrFP217K may be the sequence of SEQ ID NO: 5.

In accordance with some embodiments, the aforementioned exogenous nucleotide sequence encoding glycosyltransferase GtCGTV93Q/Y193F may include a sequence with at least 85% sequence similarity with SEQ ID NO: 6, for example, may include a sequence having at least 88%, 90%, 92%, 95%, 98% or 99% sequence similarity with SEQ ID NO: 6, but it is not limited thereto. In accordance with some embodiments, the aforementioned exogenous nucleotide sequence encoding glycosyltransferase GtCGTV93Q/Y193F may include the sequence of SEQ ID NO: 6. In accordance with some embodiments, the aforementioned exogenous nucleotide sequence encoding the glycosyltransferase GtCGTV93Q/Y193F may be the sequence of SEQ ID NO: 6.

In order to make the above-mentioned and other purposes, features and advantages of the present disclosure more thorough and easy to understand, a number of examples are given below, and are described in detail as follows, but they are not intended to limit the scope of the present disclosure.

### Example 1—Acclimating Strains to Obtain Acid-Tolerant Strains

The culture solutions of following formula (A) and formula (B) were prepared:
Formula (A): LB culture broth (Luria broth), antibiotic Ampicillin (100 mg/mL), Spectinomycin (40 mg/mL) and Kanamycin (50 mg/mL);
Formula (B): Glucose 5 g/L (can be 1-30 g/L), phosphate buffer (PB), antibiotic Ampicillin (100 mg/mL), Spectinomycin (40 mg/mL) and Kanamycin (50 mg/mL).

Escherichia coli K-12 was inoculated into a 1.5% culture medium containing formula (A) and agar, and cultured overnight at 37° C. Any three colonies in the culture medium were selected and numbered as AC1, AC2, and AC3, and cultured in 2 ml of formula (A) culture medium at 37° C. overnight. Next, acetic acid, hydrochloric acid, citric acid or salts of the aforementioned acids was added to formula (B) to prepare culture solutions of pH 6.5, pH 6, pH 5.5, pH 5, pH 4.5 and pH 4 respectively. First, the aforementioned formula (A) culture solution containing colonies was inoculated into the culture solution with pH 6.5, and was cultured at 30° C. for 24 hours. The growth of the strains was analyzed with a spectrophotometer at OD₆₀₀. After confirming that the strains have continued to grow, 10% of the strains were transferred to new culture solutions (transferred to culture media of pH 6, pH 5.5, pH 5, pH 4.5 and pH 4 in sequence) every 72 hours. The strains were cultured under the environments of each pH value for 14 days until it was observed that the strains could survive in that pH environment.

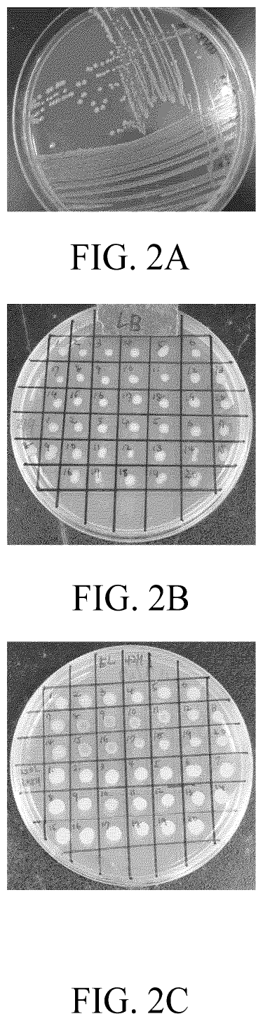

The acclimated E. coli that is tolerant to an acidic environment was obtained through the aforementioned steps, and then the strains were preserved. The preserved strains were inoculated into a 1.5% culture medium (pH=7) containing formula (A) and agar, and a 1.5% culture medium (pH=4) containing formula (A) and acetic acid, hydrochloric acid, citric acid or salts of the aforementioned acids and agar. The strains were activated and their survival rates were confirmed. The results are shown in FIG. 2A to FIG. 2C.

FIG. 2A shows that the acclimated acid-tolerant strains were activated in the culture medium (pH=7) after culturing at 37° C. for 24 hours. FIG. 2B shows the growth condition of the aforementioned acid-tolerant strains when they were transferred to a new medium (pH=4) and cultured at 37° C. for 24 hours. FIG. 2C shows the growth condition of the aforementioned acid-tolerant strains when they were once again transferred to a new medium (pH=4) and cultured at 37° C. for 24 hours. As shown in FIG. 2A to FIG. 2C, the survival rates of the acclimated strains can reach 100% after being cultured in a pH 4 environment for 24 hours, confirming that the strains can tolerate the acidic environment of pH 4.

### Example 2—Genome Evolution Analysis of the Acclimated Acid-Tolerant Strains

Next Generation Sequencing (NGS) was used to analyze the transcriptome of the acclimated strains. Under different environmental pressures, the expression changes of all genes in the genome were measured to obtain genes that may be related to the strain's ability to tolerate acidic environments. The native Escherichia coli (source Escherichia coli) and the acclimated acid-tolerant strains were cultured in the culture solutions of pH 7 and pH 4 respectively for 24 hours. The E.Z.N.A Bacterial RNA Kit was used to extract the RNA and transcribe it into DNA, and then the transcriptome data was obtained through NGS analysis. Bioinformatics software Clusters of Orthologous Groups and KEGG were then used to analyze genes with differential expression.

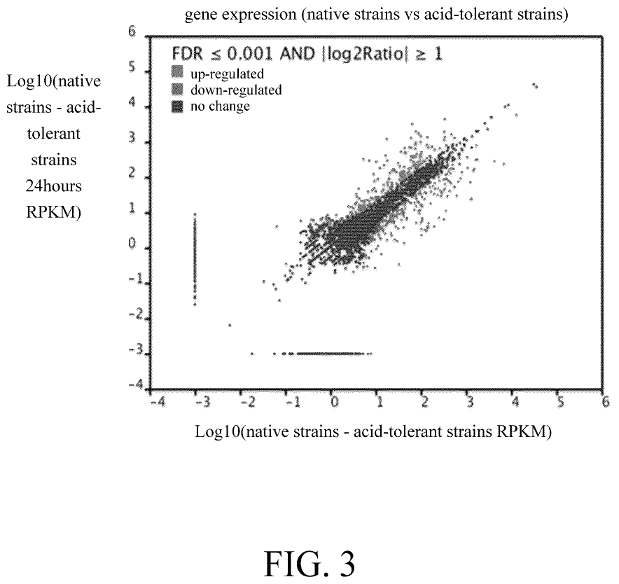

The differentially expressed genes of native Escherichia coli (cultured in a pH 7 environment for 24 hours) and acid-tolerant strains (cultured in a pH 4 environment for 24 hours) were drawn into a graph. All Unigenes were divided into three parts. The red dots represent genes with up-regulated expression (relative to native E. coli), the green dots represent genes with down-regulated expression, and the blue dots represent no change in expression. The results are shown in FIG. 3. As shown in FIG. 3, the expression of 221 genes was up-regulated and the expression of 324 genes was down-regulated. In addition, genes with high differential expression levels are listed in Table 1.

TABLE 1

| Gene ID | Log2 | Gene/product name |
|---|---|---|
| Unigene2149 | 12.61 | dsdA/Anaerobic C4-dicarboxylate transporter |
| Unigene3390 | 11.15 | dcuC/flagellar m-ring protein Flif |
| Unigene1389 | 10.38 | glaA/glucoamylase |
| Unigene1638 | 9.41 | yhjx/Uncharacterized MFS-type transporter |
| Unigene2169 | 11.68 | NifE/nitrogenase MoFe cofactor biosynthesis protein |
| Unigene1850 | 11.11 | FliA/flagellar biosynthesis sigma factor |
| Unigene1101 | 11.02 | recA/recA protein |

### Example 3—Establishment of Acetic Acid Metabolism-Enhanced Acid-Tolerant Strains

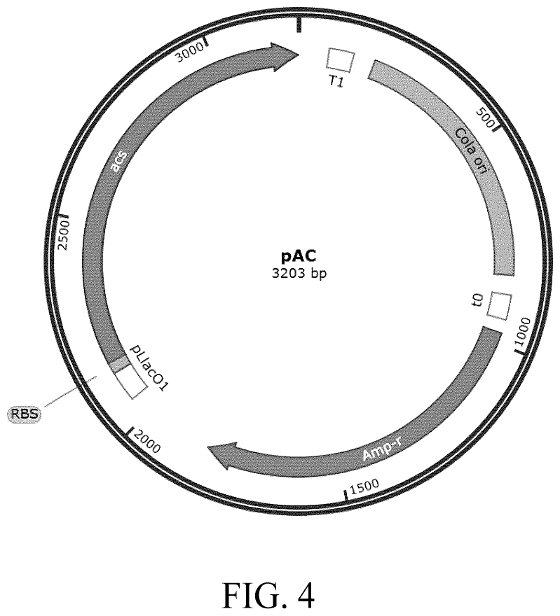

A plasmid with acetyl-CoA synthetase (acs) gene was constructed by genetic modification. In which, Escherichia coli K-12 was used as a template, a primer pair was designed to have the fragments of the target acs gene, and then PCR amplification was performed using Taq DNA Polymerase Master Mix RED. After purification by electrophoresis, a DNA fragment of the acs gene was obtained, and then restriction enzymes were used to act on the plasmid pAC as well as the DNA fragment was ligated to the plasmid. The structure of the plasmid constructed thereby with the nucleotide sequence encoding acetyl-CoA synthetase (SEQ ID NO: 1) (acs gene) is shown in FIG. 4. The plasmid carrying the acs gene was then introduced into competent E. coli cells and screened with antibiotics that met the tolerance of the plasmid. The obtained strains were further confirmed whether they contained a fragment of the acs gene using a PCR reaction. After the acs gene was introduced into the aforementioned acclimated acid-tolerant strains, the acetic acid metabolism-enhanced acid-tolerant strains according to the embodiments of the present disclosure were obtained.

### Example 4—Comparison of Growth Ability of Acid-Tolerant Strains and Acetic Acid Metabolism-Enhanced Acid-Tolerant Strains in Different Acidic Culture Solutions

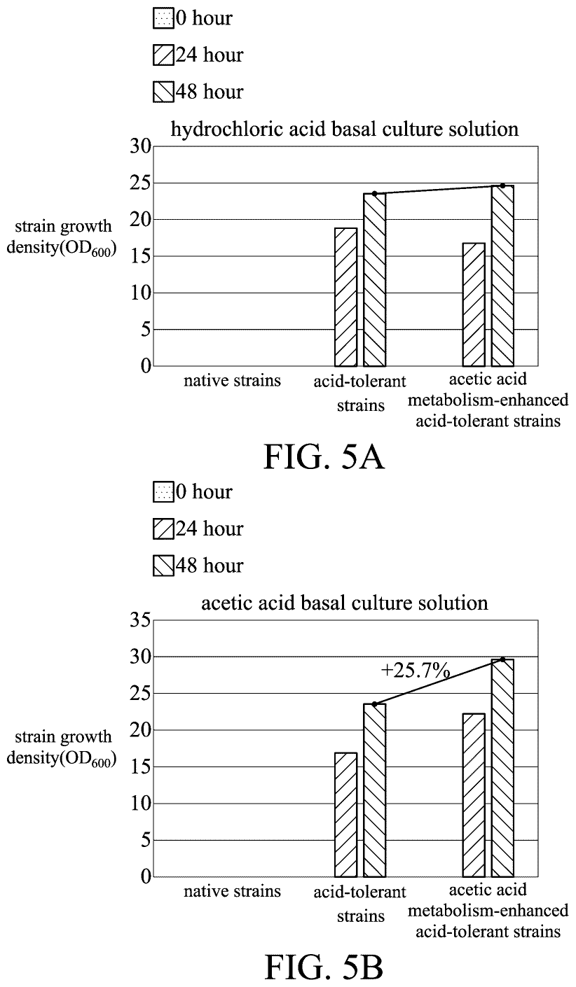

The growth conditions of native strains, acid-tolerant strains (with acid tolerance through acclimation) and acetic acid metabolism-enhanced acid-tolerant strains (with acid tolerance through acclimation and were introduced with acs gene) were compared in acidic basal culture solutions (the culture solutions were M9 acid, containing glucose 10 g/L, and were prepared with hydrochloric acid/acetic acid to adjust the pH value to pH=4). The results are shown in FIG. 5A and FIG. 5B.

FIG. 5A shows the strain growth density (measured by spectrophotometer at OD₆₀₀) of native strains, acid-tolerant strains and acetic acid metabolism-enhanced acid-tolerant strains growing in hydrochloric acid basal culture solution for 0 hours, 24 hours and 48 hours. In the hydrochloric acid basal culture solution, the native strains almost did not grow, and the growth conditions of the acid-tolerant strains and the acetic acid metabolism-enhanced acid-tolerant strains were both good. After 48 hours of culture, there was not much difference in the growth numbers of the acid-tolerant strains and the acetic acid metabolism-enhanced acid-tolerant strains.

FIG. 5B shows the strain growth density (measured by spectrophotometer at OD₆₀₀) of native strains, acid-tolerant strains and acetic acid metabolism-enhanced acid-tolerant strains growing in acetic acid basal culture solution for 0 hours, 24 hours and 48 hours. In the acetic acid basal culture solution, the growth result of acid-tolerant strains was similar to that in the hydrochloric acid basal culture solution, while the growth ability of acetic acid metabolism-enhanced acid-tolerant strains was significantly better. Compared with the acid-tolerant strains, the cell growth of the acetic acid metabolism-enhanced acid-tolerant strains increased by approximately 25.7%.

It can be seen from the foregoing results that the acid-tolerant strains and the acetic acid metabolism-enhanced acid-tolerant strains selected in the embodiments of the present disclosure can tolerate the acidic environment of pH 4. When using hydrochloric acid basal culture solution to culture acid-tolerant strains and acetic acid metabolism-enhanced acid-tolerant strains, there was little difference in the growth conditions of acid-tolerant strains and acetic acid metabolism-enhanced acid-tolerant strains because hydrochloric acid had no carbon source to use. When using acetic acid basal culture solution to culture acid-tolerant strains and acetic acid metabolism-enhanced acid-tolerant strains, the acetic acid metabolism-enhanced acid-tolerant strains can use acetic acid as a carbon source to promote growth. Compared with the acid-tolerant strains, the growth ability of the acetic acid metabolism-enhanced acid-tolerant strains was increased by about 25.7%.

### Example 5—Plasmid Construction of Carmine-Producing Strains

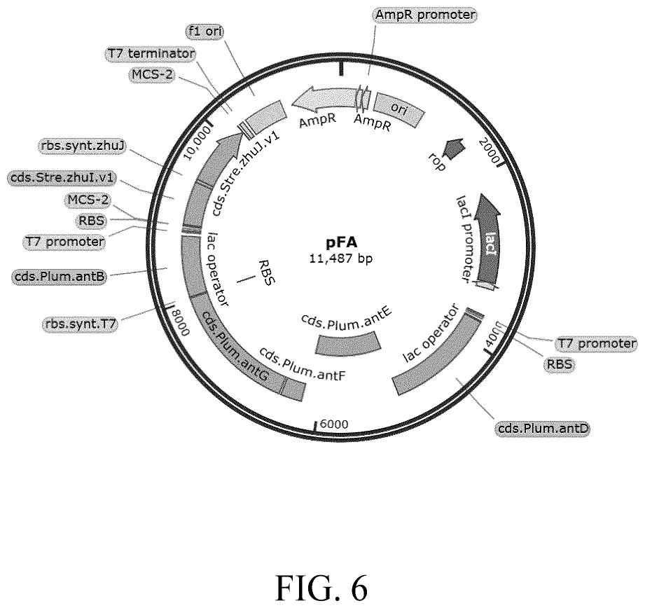

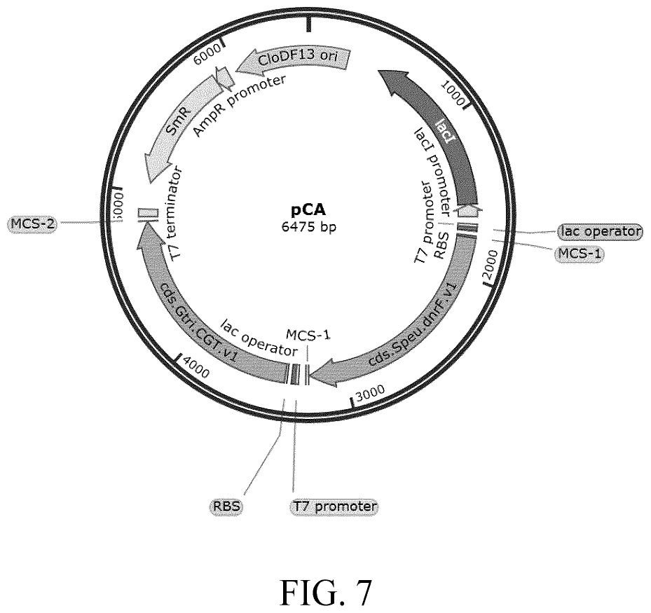

With a method similar to that of Example 3, a plasmid carrying the genes related to the synthesis of carmine was constructed by genetic modification, and the genes related to the synthesis of carmine were separately introduced into two plasmids to reduce the size of the plasmid and to improve the success rate of constructing plasmids and strains. The nucleotide sequence encoding zhuI (SEQ ID NO: 2), the nucleotide sequence encoding zhuJ (SEQ ID NO: 3) and the nucleotide sequence encoding antDEFBG (SEQ ID NO: 4) were introduced into a plasmid (pFA). The structure of the constructed plasmid carrying zhuI, zhuJ and antDEFBG genes is shown in FIG. 6. In addition, the nucleotide sequence encoding dnrFP217K (SEQ ID NO: 5) and the nucleotide sequence encoding GtCGTV93Q/Y193F (SEQ ID NO: 6) were introduced into another plasmid (pCA). The structure of the constructed plasmid carrying dnrFP217K and GtCGTV93Q/Y193F genes is shown in FIG. 7.

Single introduction of the aforementioned pFA plasmid can produce the intermediate flavokermesic acid of the biosynthetic pathway (as shown in FIG. 1), while simultaneous introduction of the pFA plasmid and pCA plasmid can produce carminic acid.

### Example 6—Establishment of Acid-Tolerant Strains and Acetic Acid Metabolism-Enhanced Acid-Tolerant Strains and Comparison of the Ability to Produce Carmine Therebetween

After simultaneously introducing carmine production plasmids (pFA and pCA) into the aforementioned acid-tolerant strains and acetic acid metabolism-enhanced acid-tolerant strains, the production of carmine in acidic basal culture solution (the culture solutions were M9 acid, containing 10 g/L glucose, and were prepared with hydrochloric acid/acetic acid to adjust the pH value to pH=4) by the acid-tolerant strain used for carmine production and the acetic acid metabolism-enhanced acid-tolerant strains used for carmine production were compared.

The quantitative analysis of carmine was performed by high-performance liquid chromatography (HPLC), and Japan GL Sciences InertSustain C18 analytical column (5 μm, 4.6×250 mm) was used for analysis. After dissolving the carmine standard in water, 6% acetate in H₂O/acetonitrile=60/40 was used as the mobile phase, the flow rate was set to 0.6 mL/min, the temperature was set to 40° C., and the analysis was performed with the detector set to UV and wavelength 460 nm.

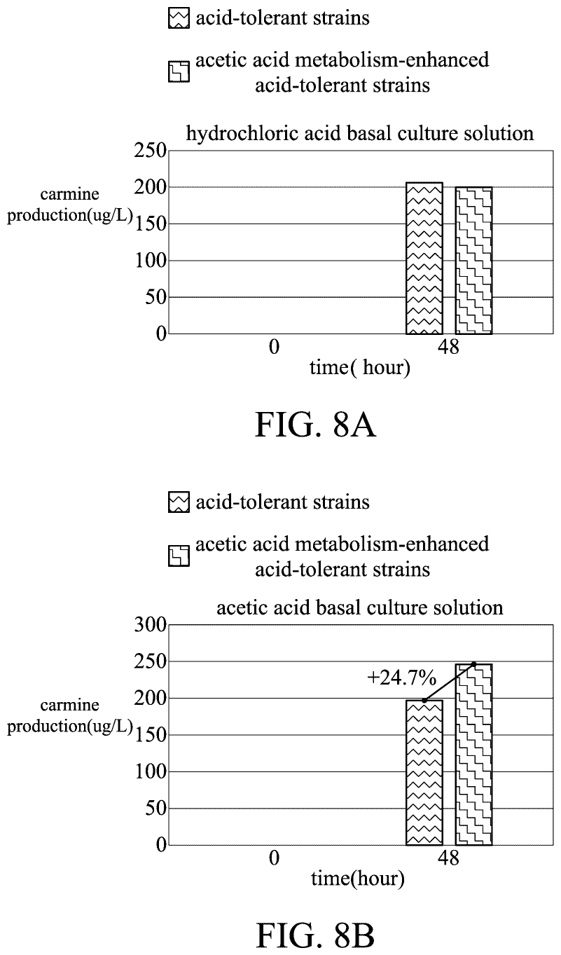

FIG. 8A shows the carmine production of the acid-tolerant strains and acetic acid metabolism-enhanced acid-tolerant strains used for carmine production in hydrochloric acid basal culture solution for 0 hours and 48 hours. In hydrochloric acid basal culture solution, there was not much difference in carmine production between acid-tolerant strains and acetic acid metabolism-enhanced acid-tolerant strains.

FIG. 8B shows the carmine production of the acid-tolerant strains and acetic acid metabolism-enhanced acid-tolerant strains used for carmine production in acetic acid basal culture solution for 0 hours and 48 hours. In the acetic acid basal culture solution, the carmine production of the acid-tolerant strains was similar to the results in the hydrochloric acid basal culture solution, while the carmine production of the acetic acid metabolism-enhanced acid-tolerant strains was significantly better than the results in the hydrochloric acid basal culture solution. In the acetic acid basal culture solution, compared with the acid-tolerant strains, the carmine production of the acetic acid metabolism-enhanced acid-tolerant strains increased by approximately 24.7%.

From the foregoing results, it can be seen that when using hydrochloric acid basal culture solution to culture the acid-tolerant strains and the acetic acid metabolism-enhanced acid-tolerant strains for carmine production, there was no carbon source available for hydrochloric acid. Therefore, there was little difference in the carmine yield between acid-tolerant strains and acetic acid metabolism-enhanced acid-tolerant strains used for carmine production. When using acetic acid basal culture solution to culture the acid-tolerant strains and the acetic acid metabolism-enhanced acid-tolerant strains for carmine production, the acetic acid metabolism-enhanced acid-tolerant strains for carmine production can utilize the acetic acid as a carbon source to promote growth and carmine production. Compared with the acid-tolerant strains used for carmine production, the carmine production of acetic acid metabolism-enhanced acid-tolerant strains increased by approximately 24.7%. Therefore, the foregoing results confirm that the acetic acid metabolism-enhanced acid-tolerant strains used for carmine production in the embodiments of the present disclosure can utilize acetic acid as a carbon source to promote carmine production.

To summarize the above, the embodiments of the present disclosure screen and establish strains that are tolerant to acidic environments and can utilize acetic acid as a carbon source, and thereby further prepare genetically engineered strains that can produce a target chemical (for example, carmine). By improving the strain's ability to convert acetic acid into metabolic raw materials for the target chemical, the acetic acid utilization rate of the strains and the yield of the target chemical can be increased.

Although some embodiments of the present disclosure and their advantages have been described as above, it should be understood that various changes, substitutions and alterations can be made herein without departing from the spirit and scope of the disclosure as defined by the appended claims. In addition, each claim constitutes an individual embodiment, and the claimed scope of the present disclosure also includes the combinations of the claims and embodiments. The scope of protection of the present disclosure is subject to the definition of the scope of the appended claims.

What is claimed is:

1. A genetically engineered microorganism, comprising a higher expression level of acid-tolerant genes compared to a source microorganism, wherein the acid-tolerant gene comprises at least one of dsdA, dcuC and glaA.

2. The genetically engineered microorganism as claimed in claim 1, further comprising:
at least one exogenous nucleotide sequence encoding acetyl-CoA synthetase (ACS).

3. The genetically engineered microorganism as claimed in claim 1, wherein the source microorganism comprises Escherichia coli, Corynebacterium glutamicum, Bacillus subtilis, Pseudomonas putida, Yarrowia lipolytica, Saccharomyces cerevisiae, or Pichia pastoris.

4. The genetically engineered microorganism as claimed in claim 1, which has tolerance to an acidic environment, wherein the pH value of the acidic environment ranges from pH 3 to pH 5.

5. The genetically engineered microorganism as claimed in claim 2, wherein the exogenous nucleotide sequence further comprises an exogenous nucleotide sequence encoding an enzyme related to the synthesis of a target chemical.

6. The genetically engineered microorganism as claimed in claim 5, wherein the target chemical comprises carmine, para-aminobenzoic acid (PABA), indigo blue, melanin, or a combination thereof.

7. The genetically engineered microorganism as claimed in claim 6, wherein the target chemical is carmine, and the enzymes related to the synthesis of carmine comprise at least one of the following:
cyclase ZhuI, aromatase ZhuJ, polyketide synthase (octaketide synthase complex antDEFBG), hydroxylase dnrFP217K, glucosyltransferase GtCGTV93Q/Y193F, glucosyltransferase UGT2, monooxygenase aptC and 4′-phosphopantetheinyl transferase npgA.

8. The genetically engineered microorganism as claimed in claim 6, wherein the enzyme related to the synthesis of the target chemical comprises glutamine aminotransferase PabA, 4-amino-4-deoxychorismate synthase PabB, 4-amino-4-deoxychorismate lyase PabC, naphthalene dioxygenase NDO, or a combination thereof.

9. The genetically engineered microorganism as claimed in claim 7, wherein the target chemical is carmine, and the deposit number of the genetically engineered microorganism is BCRC 940699.

10. A novel genetically engineered E. coli strain whose deposit number is BCRC 940699, wherein the novel genetically engineered E. coli strain has a higher expression level of an acid-tolerant gene compared to a source microorganism, and the acid-tolerant gene comprises at least one of dsdA, dcuC and glaA, and wherein the novel genetically engineered E. coli strain comprises exogenous nucleotide sequences encoding acetyl-CoA synthetase and encoding an enzyme related to carmine synthesis.

11. A method of preparing a genetically engineered microorganism, comprising the following steps:
(a) acclimating a microorganism with an acidic culture solution;
(b) introducing an exogenous nucleotide sequence encoding acetyl-CoA synthetase into the acclimated microorganism; and
(c) introducing an exogenous nucleotide sequence encoding an enzyme related to the synthesis of a target chemical into the acclimated microorganism, to obtain the genetically engineered microorganism.

12. The method of preparing a genetically engineered microorganism as claimed in claim 11, wherein the microorganism comprises Escherichia coli, Corynebacterium glutamicum, Bacillus subtilis, Pseudomonas putida, Yarrowia lipolytica, Saccharomyces cerevisiae, or Pichia pastoris.

13. The method of preparing a genetically engineered microorganism as claimed in claim 11, wherein the acidic culture solution comprises acetic acid, hydrochloric acid, citric acid, salts of the aforementioned acids, or a combination thereof.

14. The method of preparing a genetically engineered microorganism as claimed in claim 11, wherein the step of acclimation comprises:
preparing a plurality of acidic culture solutions with different pH values ranging from pH 6.5 to pH 4; and
culturing the microorganism sequentially with the culture solution with a high pH value to a low pH value from the plurality of acidic culture solutions.

15. The method of preparing a genetically engineered microorganism as claimed in claim 14, wherein the pH values of the plurality of acidic culture solutions comprise three or more of pH 6.5, pH 6, pH 5.5, pH 5, pH 4.5 and pH 4.

16. The method of preparing a genetically engineered microorganism as claimed in claim 14, wherein the plurality of acidic culture solutions with different pH values are used to culture the microorganism for 10 days to 20 days respectively.

17. The method of preparing a genetically engineered microorganism as claimed in claim 11, wherein the target chemical comprises carmine, para-aminobenzoic acid (PABA), indigo blue, melanin, or a combination thereof.

18. The method of preparing a genetically engineered microorganism as claimed in claim 17, wherein the target chemical is carmine, and the enzymes related to the synthesis of carmine comprise at least one of the following:
cyclase ZhuI, aromatase ZhuJ, polyketide synthase (octaketide synthase complex antDEFBG), hydroxylase dnrFP217K, glucosyltransferase GtCGTV93Q/Y193F, glucosyltransferase UGT2, monooxygenase aptC and 4′-phosphopantetheinyl transferase npgA.

19. A method of producing a target chemical, comprising the following steps:
(a) providing a genetically engineered microorganism as claimed in claim 1; and
(b) culturing the genetically engineered microorganism in an acidic culture solution;
(c) culturing the genetically engineered microorganism at a temperature of 15° C. to 42° C. for 8 hours to 80 hours to produce a culture solution containing the target chemical; and
(d) isolating and purifying the target chemical from the culture solution of step (c).

20. The method of producing a target chemical as claimed in claim 19, wherein the pH value of the acidic culture solution ranges from pH 3 to pH 5, wherein the acidic culture solution comprises acetic acid and/or its salts.
